(19) United States
(12) Patent Application Publication
(10) Pub. No.: US 2008/0019862 A1
(43) Pub. Date: Jan. 24, 2008

(54) Evap odor control system

(76) Inventor: Steven Forrest White, Oklahoma City, OK (US)

Correspondence Address: JERRY W. ANDERSON, 108 E. MAIN, NORMAN, OK, 73089, US

(21) Appl. No.: 11/818,404

(22) Filed: Jun. 14, 2007

Related U.S. Application Data
(60) Provisional application No. 60/813,675, filed on Jun. 14, 2006.

(51) Int. Cl. A61L 9/02; A61L 9/00; A61L 9/03; A61L 9/015

(52) U.S. Cl. 422/5; 422/123; 422/124; 422/125

(57) ABSTRACT

The invention is a apparatus and method for the suppression of offensive orders by the introduction and dispersal of water soluble deodorant/reactive compounds in the vapor phase. The apparatus can used to reduce or eliminate offensive odors emanating from cigarette smoke, and the suppression of said offensive odors in industrial applications.

This invention claims priority from previous provisional application No. 60/813675 filed on Jun. 15, 2006.

No federal funds were used in the development of this device

There is no microfiche appendix

### BACKGROUND OF INVENTION

This invention was conceived to address the problems of offensive odors in public places and work spaces. This invention relates to the field of odor control devices. This invention is a device supplementing the standard methods for the control of offensive odors.

There exists a need for an apparatus and procedure to control offensive odors in commercial enterprises, such as casinos, restaurants, paper processing plants, waste water processing plants, or fish processing plants. In some cases, the odors are due to cigarette smoke, or to materials produced inherent to the industrial process. In some cases the odorous compounds produced in the facility have been demonstrated to be actually reduced in concentration by reaction or other mechanism by the introduction of the deodorant compound.

Other procedures have been used to disperse deodorant compounds into the ambient air, however, these have been mainly oil based masking agents, or the use of misting. This procedure is based upon the use of water based deodorant compounds, which include reactive components, as well as pleasant aromas. This procedure is specifically designed to deliver the deodorant compounds in true vapor phase.

Conventional methods of odor suppression rely upon the spraying of masking agents and other chemicals in a mist, which has problems with entrained water or solvent depositing in the duct work, if using High volume air conditioning (HVAC) systems, or in the tubing used to disperse the reagents. Thus resulting in uneven and inefficient dispersion of the reagents, either too little of the reagent being delivered to the desired areas, or too much. This device releases the reagent in a true vapor form, requiring much less of the reagent, and ensuring a more even distribution of the reagent in the desired area. Deposition of water or organic solvent in duct work is eliminated or greatly reduced.

The described device delivers the reagent vapor in a controlled method, the reagents react with the offensive odor molecules in the vapor phase, and eliminate or reduce the concentrations of the offensive odor in the workspace or the public space.

The advantage of water based compounds combined with the below described apparatus yields an efficient and economical application of the aforesaid deodorant in a precise and controlled method. Delivery of the deodorant compounds into the atmosphere in true vapor phase ensures efficient dispersal of said compounds throughout the facility.

The deodorants used, may consist, but are not limited to, any one of the following commercially available mixtures, depending upon the desired application: 9037 Smoke Away, Malodor II with Citrus, Malodor II with 9028 (Spring Garden), Nitrex with 9023 (Southern Breeze) 9015 (Fresh & Clean), 9042 (Sea Breeze), 9011 (No Scent), and Nitrex Desludge. It is emphasized that these compounds are totally water soluble.

The applications of this apparatus and methodology may include but are not limited to he following: paper processing plants, fish processing plants, waste water treatments, restaurants and casinos.

The apparatus used comprises of the following; an air pump, a thermostatically controlled reservoir with aerator, and a dispersal system, tubing and duct-work, to disperse the deodorant mixture, and electronics to control the air pump flow rate and temperature of the reservoir. Said reservoir, air pump and electronics is enclosed in a cabinet. The duct-work to disperse the deodorant may consist of custom installed large diameter tubing, typically polyethylene, but may be other similar inert material, which extends throughout the area wherein odor control is desired. The tubing shall have holes drilled in the tubing to serve as dispersal points. Said dispersal point's spacing and diameter is are dependent upon the application. If the facility has existing High Volume Aid Conditioning (HVAC) system, a vapor dispenser, which is inserted into the HVAC, to disperse the deodorant into the air stream, in a vapor phase, which will then dispense said deodorant/reactant throughout the facility.

In use the air pump is connected to the reservoir and hence the dispersal system, the aforesaid tubing, ductwork or vapor dispenser, if HVAC is used. The air pump provides the desired air flow over the heated fluid in the reservoir. A small fraction of the air flow is diverted to the bottom of the reservoir, and through a bubbling system, the aerator, which keeps the solution well mixed. The dispersal system is sized to provide a back pressure for the air pump and reservoir. The amount of deodorant dispersed is dependent upon the temperature of the solution in the reservoir and the flow rate of the air over the solution. As the solution of water and deodorant in the reservoir is heated, the vapor pressure of the water and the deodorant increase, this vapor is swept by the air flow from the air pump into the dispersal system, the ducting and tubing necessary to deliver the deodorant to the desired locations. The deodorant in the vapor phase is dispersed throughout the facility. In restaurants and casinos, which have an existing HVAC, the deodorant is vented into the HVAC system, through a vapor dispenser. The vapor dispenser disperses the deodorant in the vapor form, ensuring that a controlled amount of the deodorant is delivered to the HVAC sysem.

The method for use of the mechanism described above is to charge the reservoir with a measured aliquot of water, to add the chosen deodorant mixture, seal the reservoir, to set the temperature to the desired temperature, typically 105 deg. F., allow the reservoir to equilibrate and to start the blower fan. The blower fan pressurizes the vent system, starts the aerator in the reservoir, and starts the dispersal of the deodorant vapor into the vent line. The blower fan controls are set to the specified flow rate for the application.

### DESCRIPTION OF PRIOR ART

The evolution of obnoxious odors and/or hazardous chemicals in the work space and in neighboring areas has been a problem for many years. Many attempts have been to address this problem. Usually methods have been the use of masking agents, odorous chemicals which are less objectionable to the human olfactory system. Newer methods have involved the use of reactive chemicals to remove or reduce the concentration of the obnoxious compounds. A combination of both masking agents and reactive compounds has been found to be highly effective. The following patents illustrate some of the approaches used.

U.S. Pat. No, 3,442,602, C. Diehl, issued May 6, 1969, disclosed a method for the deodorizing air in an industrial facility based upon the dispersion of water soluble agents using a high velocity air stream. In this device the water and deodorant was dispersed as a mist. This device is limited in use to

U.S. Pat. No. 3,576,593, D. Cicirello, issued Apr. 27, 1971 disclosed a method for deodorizing room air by recirculating air treated with a volatile chemical odorant.

U.S. Pat. No. 4,166,087, Cline et al, issued Aug. 28, 1979, discloses a vapor dispenser and method to treat room air with a variety of diverse compounds such as deodorants.

The common element of all of these devices is the use of water soluble compounds, and the entrainment of volatilized deodorants in an air stream into a selected area. The purpose of these devices is to remove or to neutralize odoriferous compounds in ambient air, from either industrial or residential sites. All use water soluble compounds as the reagents. All envisioned that the deodorant/water mixture was dispersed as a mist.

U.S. Pat. No. 4,444,720, H. Mayer, issued Apr. 24, 1984, discloses an apparatus for releasing an odor destroying substance into an air stream such as a kitchen exhaust hood, with a refillable reservoir open to the air stream, allowing the odor destroying compound to evaporate into the air stream. Deodorant may be a solid, gases or liquids.

U.S. Pat. No. 6,142,383, T. W. Planker, issued Nov. 7, 2000, discloses an apparatus based upon a oil-based deodorant used to control and suppress offensive odors. To differentiate between this device and the device described herein, this device is based upon a oil based deordorant, the device described herein uses water based deodorants, resulting in an increased ease of use, and expanded availability of deodorant/masking/reactive systems.

More recently a series of patents were issued to S. Dillenback, 2003 to present, U.S. Pat. Nos. 6,553,651, 6,766,651, and US App Pub. 2003/0230091, which disclosed a computer controlled system to introduce the deodorant compounds into the HVAC system, although the patents were issued only for the computer control system, the patents discussed the use of the reservoir/water based compounds.

None of these previously issued devices envisioned the use of a water based deodorant/reactive compound mixture delivered to the desired location in a true vapor phase.

### BRIEF SUMMARY OF INVENTION

This invention dispenses a mixture of reactive compounds and odorants in to the air in work spaces. The reactive compounds react with certain odorous compounds in the air, reducing their concentration and negative effect on people in the work space. Odorants assist in masking objectionable odors.

When used in the conventional manner. this invention releases both masking agetns and reactive compounds in to the work space, thus decreasing complaints by people in the work spaces regarding objectionable odors.

### BRIEF DESCRIPTION OF DRAWINGS

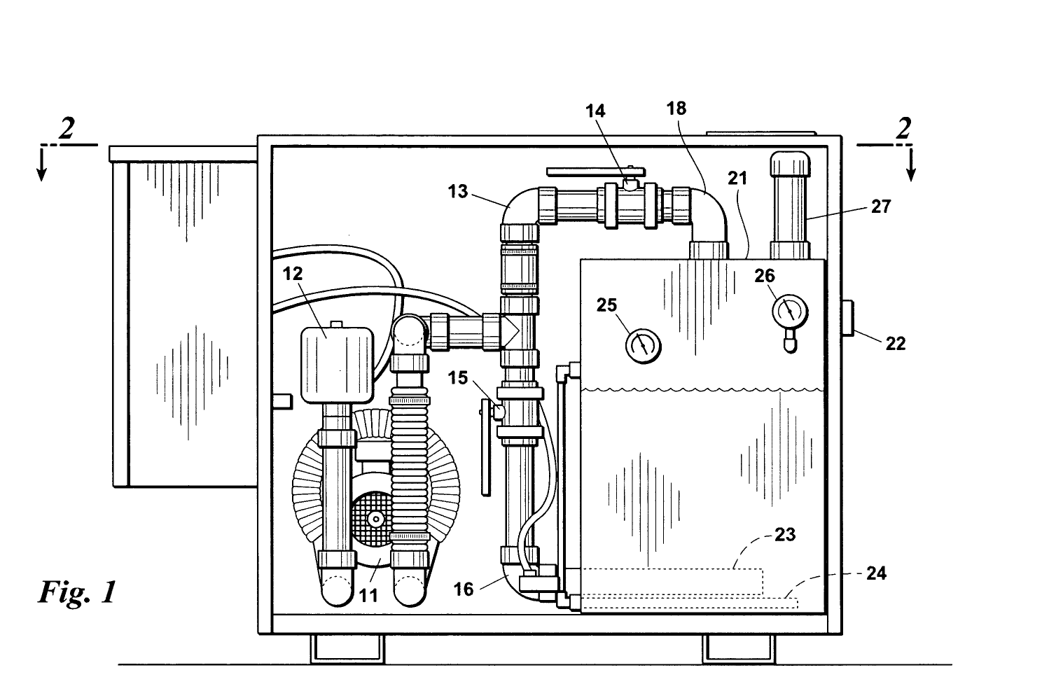

1. FIG. 1. Side view of the apparatus with covers removed.

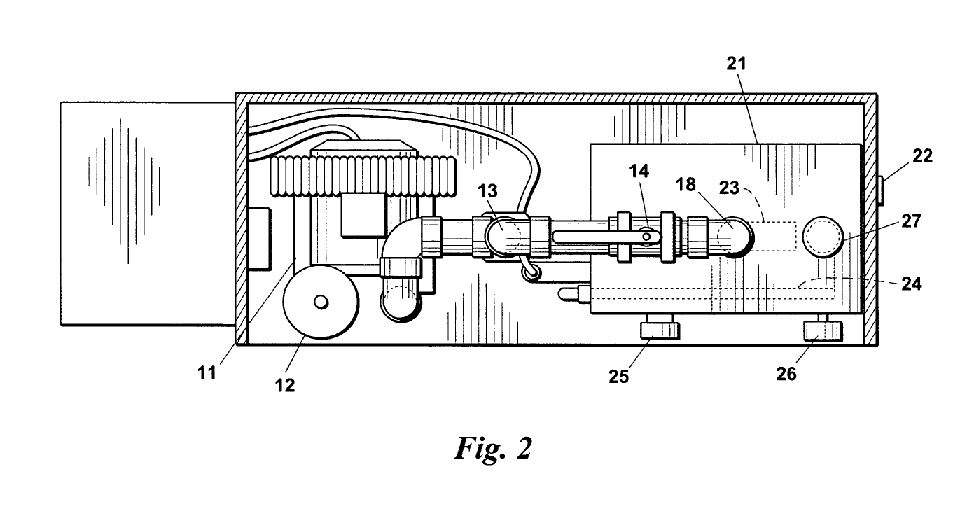

2. FIG. 2. Top view of the apparatus with cover removed

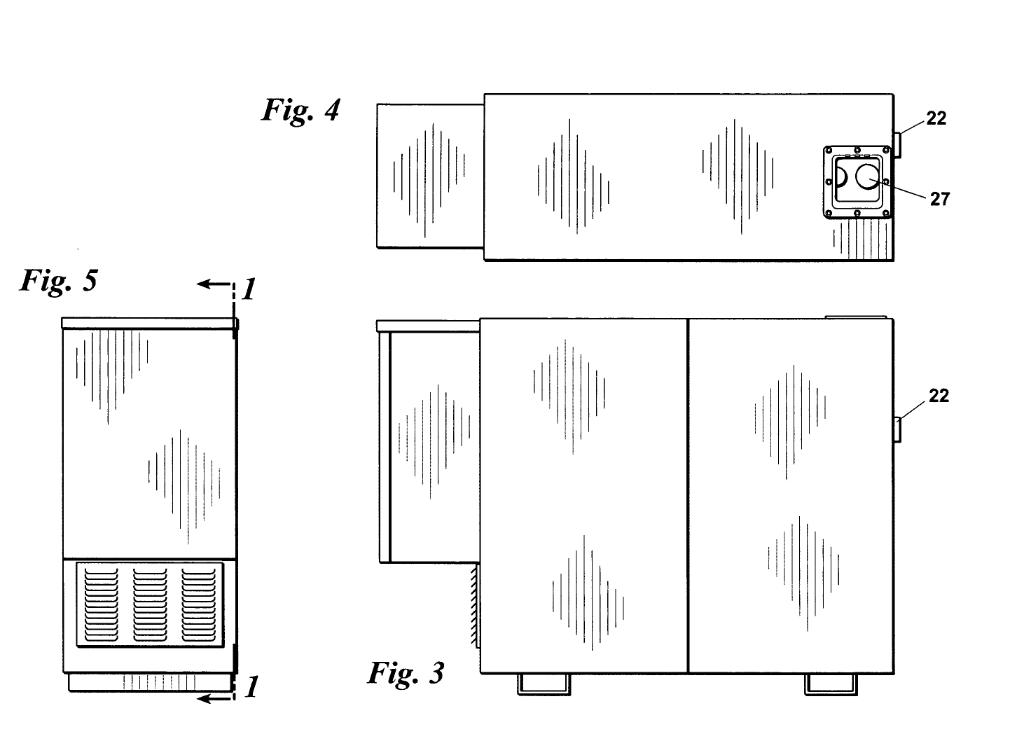

3. FIG. 3 Left side of the apparatus

4. FIG. 4. Top view of the apparatus with covers in place.

5. FIG. 5. End view of the apparatus with covers in place.

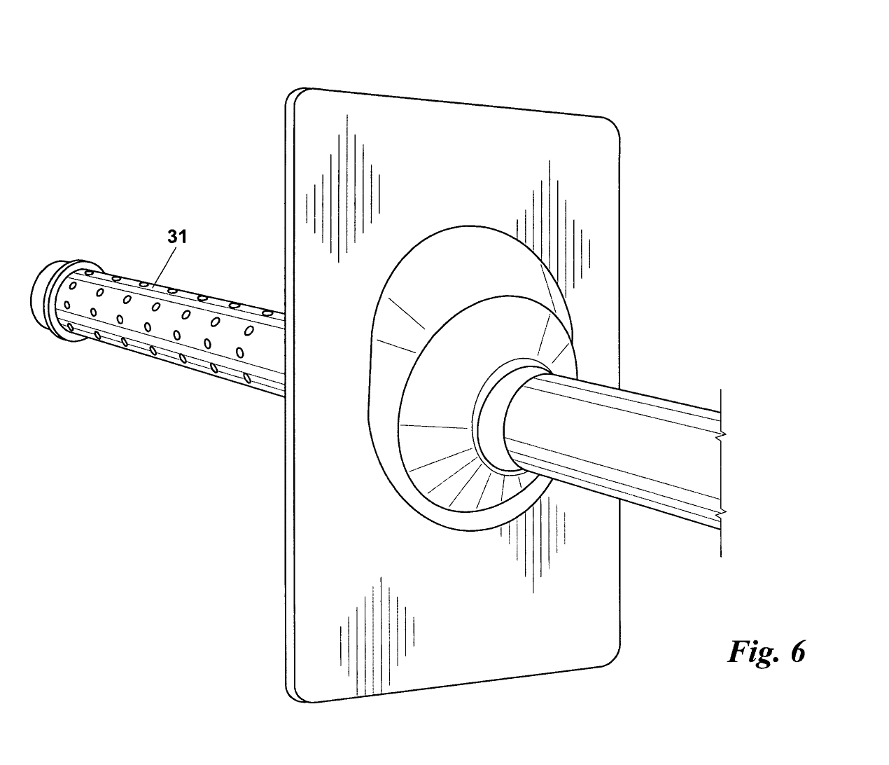

6. FIG. 6. Diagonal view of the vapor dispenser.

### DETAILED DESCRIPTION OF INVENTION

The invention herein is an apparatus designed to deliver a mixture of deodorants and reactive compounds, in a true vapor phase, into the air stream of a High Volume Air Conditioning system (HVAC), or into a distribution system to disperse the deodorant mixture in the work space.

Referring to the attached drawings, the apparatus described herein, comprises an air intake filter 12, lower air control valve 15, upper air control valve 14, reservoir 21, temperature gauge 25, pressure gauge 26, product outlet to distribution system 22, regenerative blower 11, aerator 23, and tubing 13, 16. The distribution system comprises either a vapor dispenser 31 for introduction into the High Volume Air Conditioning system, or a series of tubing, typically polyethylene, for the dispersal of the product to selected areas of the workspace.

The regenerative blower 11, controlled by the variable speed switch located within the Electronics cabinet, draws air in through the filter 12, and sends it through the tubing 13 to the air control valves, upper 14, and lower 15, typically 10% of the air is directed through tubing 16 to the aerator 23, located in the bottom of the reservoir 21. The air flow through the aerator, increases the amount of vapor evolved from the solution, and thus increases the amount of solute entrained in the air flow through the reservoir. The majority of the air flow is directed through tubing 16 to the upper air control valve 17, and thence through tubing 18 into the reservoir 21. This air flows into the reservoir and over the water deodorant mixture, entraining the vapors of the deodorant mixture, and exits the reservoir through the outlet 22 to the distribution system, to either the vapor dispenser 31, or the series of tubing for dispersal of the product.

The temperature of the reservoir is controlled by the heating element 24 located in the bottom of the reservoir, as set by the temperature control, located in the electronics cabinet. Temperature readout is by the temperature gauge 25, located on front of the reservoir. A typical temperature is 105 deg Fahrenheit. Temperature may be varied depending on the application.

The pressure in the reservoir is measured by the Pressure gauge 26, located on the front of the reservoir. The pressure is dependent upon the type of the distribution system use, and is controlled by varying the amount of air flow from the regenerative blower 11, and can be changed using the variable speed switch located in the electronics cabinet

The reservoir 21 can be filled by use on inlet port 27.

1. I claim an instrument for the introduction of water soluble vapor phase odor reactive compounds into a workspace and the method for doing same.

2. The instrument in claim 1, wherein the
instrument comprises a regenerative air pump, a thermostatically controlled reservoir, and a dispersal system, tubing and duct-work, to disperse the deodorant/reactive compound, and electronics to control the air pump flow rate and temperature of the reservoir.

3. The instrument in claim 2, wherein the reservoir has an aerator, a metal tube with a plentitude of small holes, located in the bottom of said reservoir, which emits a multitude of small bubbles into the solution in the reservoir, increasing the rate of evaporation of the reactive compounds.

4. The instrument in claim 2, wherein the thermostatically controlled reservoir has an heater, a commercially available heating element, to heat the contents of the reservoir.

5. The instrument in claim 2, wherein the regenerative air pump consists of a commercially available blower fan which supplies a controlled amount of air, to the reservoir and to the aerator, and thence to the dispersal system, said air pump being controlled by a commercially available speed limiting switch

6. The instrument in claim 2, where the dispersal system for introduction in an High Volume Air Conditioning (HVAC) system is a vapor dispenser, which comprises tubing to connect into the air conditioning duct, and a tubing with a multitude of vents therein to release the vapor into the air stream.

7. The instrument in claim 2, wherein the dispersal system for servicing a large area without using the HVAC system, comprises tubing, and vents sufficient to disperse the deodorant/reactive compound over the chosen area.

8. The instrument in claim 2, wherein the electronics necessary to control the air flow and the temperature are commercially available equipment.

9. The procedure for using the apparatus as described, comprises the following steps: the reservoir is charged with a mixture of water and the desired deodorant/reactive mixture, the temperature of the reservoir is set to the desired temperature, and the air flow through and over the water/compound mixture is set to the desired rate.

10. The procedure as per claim 9, wherein the deodorant/reactive mixture is a commercially available product, # 9037 Smoke Away, and the application is the suppression of cigarette smoke in commercial recreational areas, such as casinos.

11. The procedure as per claim 9, wherein the deodorant/reactive mixture is a commercially available product, Malodor II with Citrus, and the application is the suppression of tobacco smoke in a recreational workspace areas, such as a casino.

12. The procedure as per claim 9, wherein the deodorant/reactive mixture is a commercially available product, Malodor II with 9028 (Spring Garden), and the application is the suppression of offensive orders in commercial workspace areas, such as gyms, and indoor athletic sports venues.

13. The procedure as per claim 9, wherein the deodorant/reactive mixture is a commercially available product, Nitrex with 9023 (Southern Breeze), and the application is the suppression of offensive odors in commercial workspace areas, such as compost facilities.

14. The procedure as per claim 9, wherein the deodorant/reactive mixture is a commercially available product, Nitrex Desludge, and the application is the suppression of offensive odors in commercial workspace areas, such as a waste water treatment plants.

15. The procedure as per claim 9, wherein the deodorant/reactive mixture is a commercially available product, Nitrex with 9042 (Sea Breeze), and the application is the suppression of offensive odor in commercial workspace areas, such as a fish processing plant.

16. The procedure as per claim 9, wherein the deodorant/reactive mixture is a commercially available product, Nitrex with 9011 (No Scent), and the application is the suppression of offensive odors in commercial workspace areas, such as compost or garbage transfer facilities.

17. The procedure as per claim 9, wherein the deodorant/reactive mixture is a commercially available product, Nitrex with 9015 (Fresh & Clean), and the application is the suppression of offensive+in commercial workspace areas, such as common areas in apartment complexes.
